(19) United States
(12) Patent Application Publication
(10) Pub. No.: US 2023/0116478 A1
(43) Pub. Date: Apr. 13, 2023

(54) Improved Pathogen Deactivating Ultraviolet Light Systems and Methods

(71) Applicants: R. Hunter Knight, Houston, TX (US); David Bennett, Richmond, TX (US); Scott Johnson, Scottsdale, AZ (US)

(72) Inventors: R. Hunter Knight, Houston, TX (US); David Bennett, Richmond, TX (US); Scott Johnson, Scottsdale, AZ (US)

(21) Appl. No.: 18/046,493

(22) Filed: Oct. 13, 2022

Related U.S. Application Data
(60) Provisional application No. 63/255,080, filed on Oct. 13, 2021.

(51) Int. Cl. A61L 2/10; A61L 9/20

(52) U.S. Cl. CPC A61L 2/10; A61L 9/20; A61L 2202/11

(57) ABSTRACT

An improved pathogen deactivating ultraviolet light system and methods of use. UV light is used to deactivate aerosol pathogens, which can travel in the upper air spaces within internal spaces, such as office spaces, warehouses, and most other structures. There is a great and imperative need and desire to reduce and eliminate airborne and aerosol pathogens in the world today. The area and volume upon which some embodiments of the present invention will project the deactivating UV light are known as a “Barrier Zone.” The present invention is a novel and improved manner of creating such a Barrier Zone.

### CROSS-REFERENCE TO RELATED APPLICATIONS

The present application claims benefit to U.S. Provisional Patent Application 63/255,080 filed on Oct. 13, 2021.

### STATEMENT REGARDING FEDERALLY SPONSORED RESEARCH

Not applicable.

### BACKGROUND

The present invention generally relates to improved ultraviolet light pathogen deactivation systems and methods of use.

### SUMMARY

In the preferred embodiment of the present invention, the present invention is an improved ultraviolet light system and method to deactivate aerosol pathogens sensitive to UV light.

In several embodiments, the present invention comprises a novel system design and method of providing pathogen deactivating ultraviolet light for air to deactivate and mitigate harmful pathogens suspended in air within a room or airway system.

In several embodiments, the present invention may incorporate a down lighting fixture providing visible or decorative lighting in addition to its pathogen deactivating UV lighting.

Some embodiments of the invention may include an improved pathogen deactivating ultraviolet light system comprising: an upper mounting body having a substantially circular shape and having an upper surface; a plurality of mounting brackets disposed on said upper surface of said upper mounting body; plurality of ultraviolet lamps fixedly attached to said plurality of mounting brackets; a plurality of reflectors disposed on the upper surface of said upper mounting body and under said plurality of ultraviolet lamps; wherein, said plurality of ultraviolet lamps are arranged in a radial pattern wherein a proximal end of each ultraviolet lamp begins near a middle point of said upper mounting body and a distal end of each ultraviolet lamp is disposed toward an exterior edge formed by a perimeter of said upper mounting body; wherein, said plurality of ultraviolet lamps when energized produce a Barrier Zone upward and away from said upper surface of the upper mounting body, wherein said Barrier Zone is the active zone for the plurality of ultraviolet lamps to deactivate one or more pathogens; and wherein, said upper mounting body has a collar that effectively blocks any UV light emitted by the ultraviolet lamps from reaching any humans below the plane of the upper body of the assembly and that may be modified to block UP light projection on to specific areas above the plane of the upper body of the assembly.

Some embodiments of the present invention may include an improved pathogen deactivation ultraviolet light system comprising: a fixture having a surface, wherein said surface comprises one or more reflective surfaces, wherein said fixture further comprises a collar; a lamp receptacle for receiving a plurality of ultraviolet lamps, said lamp receptacle having sockets for receiving a terminal end of each of said plurality of ultraviolet lamps, wherein said plurality of ultraviolet lamps are disposed below said collar; a lower body comprising a plurality of support fins, sensors, processors, and lamp drivers disposed within a cavity formed by the lower body; and a power source operatively connected to the system to provide power to said lamp receptacle, said plurality of ultraviolet lamps, said sensors, said processors, and said lamp drivers.

A method for deactivating pathogens comprising the steps of: creating a barrier zone by powering a plurality of ultraviolet lamps using a system comprising: a fixture having a surface, wherein said surface comprises one or more reflective surfaces; a lamp receptacle for receiving the plurality of ultraviolet lamps, said lamp receptacle having sockets for receiving a terminal end of each of said plurality of ultraviolet lamps; a lower body comprising a plurality of support fins, sensors, processors, and lamp drivers disposed within a cavity formed by the lower body; and a power source operatively connected to the system to provide power to said lamp receptacle, said plurality of ultraviolet lamps, said sensors, said processors, and said lamp drivers.

### BRIEF DESCRIPTION OF THE DRAWINGS

For a more complete understanding of the present disclosure and the advantages thereof, reference is now made to the following descriptions to be taken in conjunction with the accompanying drawings describing specific embodiments of the disclosure, wherein:

The accompanying figures illustrate various orientations and depictions of several embodiments of the present invention.

Several of the accompanying figures illustrate exploded depictions of several embodiments of the present invention to show various parts and their orientation with other parts of the several embodiments.

FIG. 1 depicts a side profile view of one embodiment of the present invention.

FIG. 2 depicts an isometric, top-down view of one embodiment of the present invention.

FIG. 3 depicts an isometric, down-top view of one embodiment of the present invention.

FIG. 4 depicts an isometric, cut-away view of the underside of one embodiment of the present invention.

FIG. 5 depicts an exploded side profile view of one embodiment of the present invention.

### DETAILED DESCRIPTION

One or more illustrative embodiments incorporating the invention disclosed herein are presented below. Applicant has created a revolutionary and improved ultraviolet light pathogen deactivation system and method of use.

In the following description, certain details are set forth such as specific quantities, sizes, etc. to provide a thorough understanding of the present embodiments disclosed herein. However, it will be evident to those of ordinary skill in the art that the present disclosure may be practiced without such specific details. In many cases, details concerning such considerations and the like have been omitted because such details are not necessary to obtain a complete understanding of the present disclosure and are within the skills of persons of ordinary skill in the relevant art.

Referring to the drawings in general, it will be understood that the illustrations are for the purpose of describing embodiments of the disclosure and are not intended to be limiting thereto. Drawings are not necessarily to scale, and arrangements of specific units in the drawings can vary.

While most of the terms used herein will be recognizable to those of ordinary skill in the art, it should be understood, however, that when not explicitly defined, terms should be interpreted as adopting a meaning presently accepted by those of ordinary skill in the art. In cases where the construction of a term would render it meaningless, or essentially meaningless, the definition should be taken from Webster's Dictionary 2020, 11^{th} Edition. Definitions and/or interpretations should not be incorporated from other patent applications, patents, or publications, related or not, unless specifically stated in this specification, or if the incorporation is necessary for maintaining validity. As utilized herein, the following terms have the following definitions.

As used herein, “irradiance” means “the flux of radiant energy per unit area”. Irradiance is commonly expressed in terms of watts per square meter (W/m\hat{ }2). This is equivalently, a Dose Rate.

As used herein, “deactivate” or “deactivation” or “inactivate” or “inactivation” means preventing the replication of any susceptible pathogen by damaging its ability to replicate after it has entered a host cell.

As used herein, “pathogens” can include viruses, bacteria, and fungi.

As used herein, “aerosol pathogen” means a pathogen that can be suspended in air and/or freely float in the air. Typically, small enough to float in the sir means less than 1 μm in diameter.

As used herein, “aerosol pathogen” means a pathogen that can be suspended in air with or without the need to be encapsulated within a droplet.

As used herein, “droplet pathogen” means a pathogen that is encapsulated within a droplet of mucus, saliva, water, or other liquid or semi-liquid material.

As used herein, “IR” means “infrared”.

As used herein, “UV” means “ultraviolet”.

As used herein, “flux” means “the rate of flow of radiant electromagnetic energy across a given area.

As used herein, “Barrier Zone” or “BZ” means an area or volume through which flux is projected.

As used herein, “air velocity” means the direction and distance traveled per unit of time and is commonly expressed in terms of feet per minute (“FPM”) or cubic feet per minute (CFM).

As used herein, “vector anemometers” means “a device used for measuring wind or air velocity, i.e., speed and direction.” at a point.

As used herein, “COTS” means “commercial off the shelf.”

While preferred embodiments have been shown, and described, modifications thereof can be made by one skilled in the art without departing from the scope or teaching herein. The embodiments described herein are exemplary only and are not limiting. Many variations and modifications of the system and apparatus are possible and will become apparent to those skilled in the art once the above disclosure is fully appreciated. For example, the relative dimensions of various parts, the relative shapes of various parts, the materials from which the various parts are made, and other parameters can be varied.

UV light is used to deactivate aerosol pathogens, which can travel in the upper air spaces within internal spaces, such as office spaces, warehouses, and most other structures. There is a great and imperative need and desire to reduce and eliminate airborne and aerosol pathogens in the world today. The area and volume upon which some embodiments of the present invention will project the deactivating UV light are known as a “Barrier Zone.” This light must possess sufficient UV irradiance at specific wavelengths, in such BZs, so that, when absorbed by specific molecular components of pathogens, the pathogens will be deactivated. The components of the pathogens can include: DNA, RNA, proteins and other pathogen specific compounds. To deactivate a specific pathogen, the projected UV light must must be of sufficient dose rate (sufficient irradiance) over an exposure time interval, at the required wavelengths, for the damage to the target pathogens or molecules thereof deactivates.

Irradiance level is not the only requirement for deactivation or destruction. exposure duration is also a factor in the deactivation of pathogens. Similarly, sun burn is not just a factor of the strength of exposure to UV light, but also the length of time of exposure the UV light. Short exposure duration to strong sunlight may not result in sunburn, whereas longer exposure duration to lower strength sunlight can result in sunburn.

Further complicating the duration factor for pathogens to UV light exposure within interior spaces is that aerosolized pathogens can move within air currents around the interior space. The air current velocity can vary with HVAC operation, external air flows, natural air currents, movement of fans, objects, or people. Temperature variations can also affect the air currents and movement of the aerosolized pathogens. These air currents can vary over time spans of minutes, hours, days, or seasons. These air currents move the aerosolized or airborne pathogens through the upper air of the internal space and the Barrier Zone of the present invention, thereby subjecting the aerosolized or airborne pathogens to varying levels of UV irradiance.

To adjust the UV irradiance to ensure destruction or deactivation of the pathogens, the present invention requires the measurement of air currents and adjustment the UV irradiance to provide sufficient exposure duration and strength of UV light. Therefore, some embodiments of the present invention will monitor air velocity at standard and site-specific locations within an internal space's upper-air spaces. Some embodiments may construct one or more static or dynamic multi-axis velocity maps of the upper-air movement, which may be used as part of the calculation of the path of an aerosolized or airborne pathogen, including travel time intervals along a path in the Barrier Zone.

Some embodiments of the present invention can utilize sensors that measure UV flux projected from the lamps into the upper air spaces of the internal space, e.g. the Barrier Zone. These sensors can be used in conjunction with vector anemometers to calculate the minimum power required to fully destroy or deactivate an aerosolized pathogen. Some embodiments of the present invention include sensors and processors and algorithms executed by such sensors and processors to calculate the dose rate applied at points throughout the Barrier Zone of the upper air space. This value is the minimum required to fully destroy or deactivate an airborne pathogen.

Some embodiments of the present invention can include Internet connectivity to allow utilization of a central database to update system operational parameters. This can allow some embodiments of the present invention to adapt to changing environments.

Some embodiments of the present invention can utilize specialized and/or COTS sensors to monitor the Barrier Zone. The sensors may include, but not limited to, multi-axis air speed sensors, solar blind irradiance sensors, motion sensors for motion external to the lamp fixture, lamp fixture level sensors, current and voltage sensors, humidity sensors, and temperature sensors. It should be appreciated that some of these sensors may be utilized individually, combined into a single unit, or spread across multiple units to ensure coverage and provide redundancy.

Some embodiments of the present invention may include sensors to measure lamp power consumption and processors executing algorithms to track lamp ageing patterns. Some embodiments can adjust power remotely to ensure expected output levels are satisfied. It should be appreciated that in real world applications that lamps degrade over time and with usage; however, increased power to the lamp can compensate for reduced flux output by the lamp.

Some embodiments of the invention may include level sensors to monitor and ensure that the fixture housing the lamps is level and plumb, thereby ensuring that the fixture is parallel with the horizon. This feature can serve as safety function to ensure UV radiation exposure is not directed below the fixture and affect people or animals with inadvertent UV exposure. All embodiments incorporate a collar around the lamps to prevent down projection of UV light and to minimize diffraction effects.

Some embodiments of the present invention can utilize sensors to ensure UV radiation does not project below the level of the lamps in the assembly. This helps prevent inadvertent UV exposure to persons occupying the internal space containing some embodiments of the present invention. It is important to limit or avoid inadvertent UV exposure to persons or animals to minimize risks associated with UV exposure, such as “sun burn” or various forms of skin cancer. For these reasons, it is important to ensure that UV light exposure is minimized to persons or animals occupying the internal space where some embodiments of the present invention are operating.

Some embodiments of the present invention can incorporate motion or proximity sensors can be used in conjunction with some embodiments of the present invention to temporarily dim or shut off the UV light delivered by some embodiments of the present invention, while the internal space is occupied by persons or animals. Such sensors may include, but are not limited to, passive IR and/or Doppler RADAR sensors.

Some embodiments of the present invention may include sensors, monitors, and/or processors executing algorithms to measure lamp output and power usage and estimate the useful time remaining of one or more lamps. These estimates can be based on an average life span of a lamp or customized to use real world measurements.

Some embodiments of the present invention may include sensors, monitors, and/or processors executing algorithms to detect and create reports of abnormal conditions. Such reports can be delivered over a wired or wireless local area network, an Internet connection, Bluetooth, or other known or similar types of communication of electronic information. Such reports of abnormal conditions can be analyzed by maintenance or engineers owning or operating the system. Additionally, such reports can be analyzed by engineers of the manufacturer or other persons collecting such reports. Users of some embodiments of the present invention can be notified of any abnormal condition so that corrective actions may be taken. Such actions could be automatically taken by some embodiments of the present invention without the need for user intervention. Such reports can also be collected from a plurality of systems to allow for analysis of abnormal conditions, anomalies, or errors within such systems or algorithms being executed by processors contained within such systems. Some embodiments of the present invention may include additional sensors remote from the system to allow for redundancy of measurements to compare with sensors or measurements taken at or by the system. Additional sensors and measurements can reduce or eliminate potential false alarms through redundancy or faulty sensors.

Some embodiments of the present invention can provide remote diagnostics and control via Internet connectivity. Some embodiments of the present invention allow for sensor information to automatically be uploaded to the “cloud” via Internet connectivity allowing for a central server to collect and analyze sensor data. Such collected data can be continuously or intermittently verified against sensor data from remote sensors and measurements taken at the same location of the system. Aerosolized or airborne pathogen load may be estimated from such data to improve utilization of the system for individual users or across a plurality of systems and/or users via Internet connectivity.

Some embodiments of the present invention may include lamps disposed in a radial pattern, allowing for overlapping coverage with uniform illumination.

Some embodiments of the present invention may include lamps disposed in a polygonal arrangement which may can be useful in large open spaces where fixture diameter is not a limiting factor.

Some embodiments of the present invention may include one or more reflector elements having a profile that provides maximum uniform coverage and dispersion of light emitted by the lamps. Such placement would preferably be between the lamp and the Barrier Zone. Further, in some embodiments of the present invention the one or more reflector elements can be integrated into the mechanical fixture structure and provide rigidity to the fixture's structure. Preferably, the one or more reflector elements is coated or finished with a highly reflective anodized aluminum reflector material, such as Alnod™.

Some embodiments of the present invention can include an electronics package that may contain lamp drivers, communications circuits, processors that measure safety, command, control, and monitoring equipment and sensors. Some embodiments dispose the electronics package in a substructure at the bottom center of the fixture and can be readily removable for service, repair, or replacement.

Some embodiments of the present invention can include an electronics package installed away, but still connected to by wire or wireless, from the fixture.

Some embodiments of the present invention can include a down-lighting fixture providing visible light to the space or decorative ornamentation.

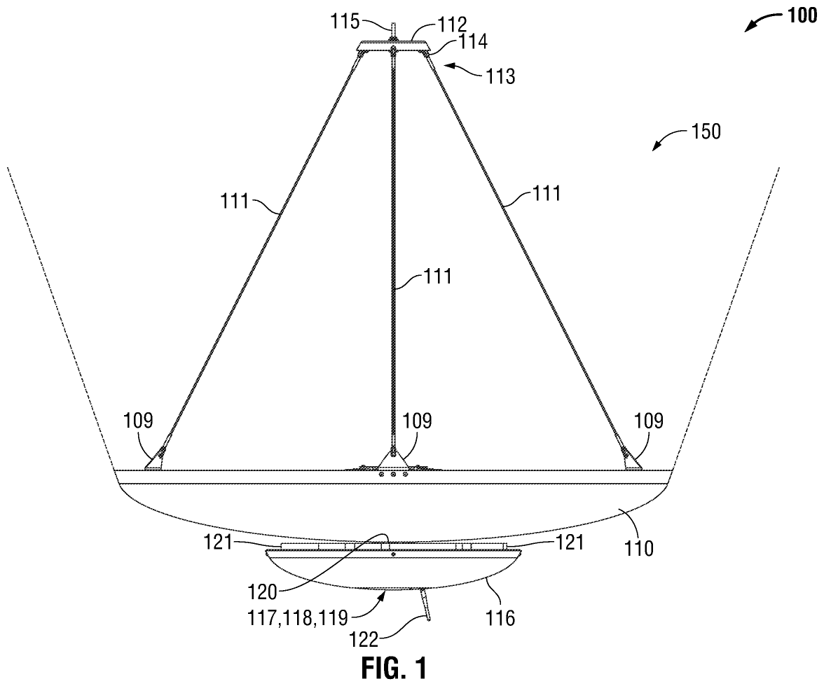

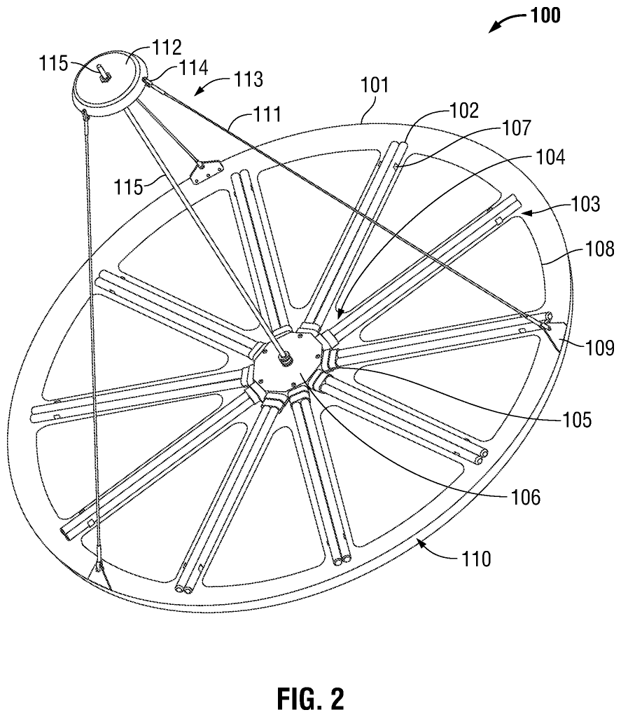

FIG. 1 illustrates one embodiment of the present invention. The system 100 depicted in this embodiment includes a fixture 101 having a circular shape for housing the lamps 102 (FIG. 2). It should be appreciated that the fixture 101 may form a collar wherein said collar extends above the lamps 102, so that when energized the lamps do not transmit ultraviolet light laterally away from the fixture and instead the ultraviolet light is projected upwards and away. It should be appreciated that the fixture can be formed in various shapes whether for functional or aesthetic purposes. It should be appreciated that various configurations of lamps 102, lamp receptacles 106, and fixtures 101 can be made to achieve varying coverage of the Barrier Zone 150 and/or for aesthetic purposes. The Barrier Zone 150, as defined herein, is represented through the use of dashed lines extending up and away from the fixture and represent the volume which the ultraviolet light is projected. The embodiment shown includes a support 115, e.g. wire or column, extending through and from the lamp receptacle 106 and perpendicular to the pattern of lamps 102 and surface of the fixture 110. The embodiment shown is intended to be mounted to a ceiling or affixed to another device to allow for the lamps to create a Barrier Zone 150 towards a ceiling or upper air space of a room. It should be appreciated that other embodiments can be mounted to poles or hang from poles or other objects affixed to a wall and achieve the same desired effect of creating a Barrier Zone 150. The embodiment shown further includes support tabs 109 affixed to a portion or surface of the fixture 101 and further include support wire 111 or other material extending from the support tab 109 to an upper support body 112 having complimentary support tabs 114 or portions or surfaces to accept a terminal end of the support wire 113 or other material. The support 115 extending through and from the lamp receptacle 106 can extend through and to the upper support body 112 to provide an attachment or mounting point for the system to a ceiling or other object. It should be appreciated that the support 115 can comprise or provide a route for electrical wire for power delivery to the system 100. It should also be appreciated that the electrical wire or power delivery could be achieved via one or more of the support wires 111 or other material. The system 100 as shown in this embodiment includes a lower body 116 disposed below the fixture wherein the sensors 117, processors 118, lamp drivers 119, antenna 122 and other electrical components can be housed separate from the lamps 102. It should be appreciated that antenna 122 could be internal or protruding from an exterior surface of the system. It should also be appreciated that antenna 122 could be a wi-fi antenna, cellular antenna, a sensor (as described herein), or combination of components for taking measurements and/or communications, such as Wi-Fi, cloud networking, or Internet access to a remote server or computer. In this embodiment, the lower body 116 is affixed to the fixture through an intermediate support 120 having a plurality of support fins 121 extending away from the intermediate support 120 in a “radial” pattern. The support fins 121 can provide rigidity to the fixture, act as heat sinks, and/or provide aesthetic appeal.

FIG. 2 illustrates one embodiment of the present invention. The system 100 depicted in this embodiment includes a fixture 101 having a circular shape for housing the lamps 102. It should be appreciated that the fixture can be formed in various shapes whether for functional or aesthetic purposes. The fixture 101 can include indentions or tracks 103 wherein one or more lamps 102 can be recessed. The fixture 101 depicted includes lamps 102 having a terminal end 104 with a socket 105 to be plugged into a lamp receptacle 106. The other end of the lamp may be secured with a clip 107, as shown, or rest on top of a portion of the fixture. The lamp receptacle 106 is disposed at or near the center of the fixture in this embodiment to create a “sunburst” or “radial” pattern of lamps 102. It should be appreciated that various configurations of lamps 102, lamp receptacles 106, and fixtures 101 can be made to achieve varying coverage of the Barrier Zone 150 and/or for aesthetic purposes. Preferably, the fixture 101 includes one or more reflectors 108 under the one or more lamps 102 to reflect additional UV light away from the surface of the fixture 101. The use of reflectors 108 helps avoid any wastage of the UV light by redirecting additional light toward the Barrier Zone, that might otherwise be absorbed by the surface of the fixture 110. The embodiment shown includes a support 115, e.g. wire or column, extending through and from the lamp receptacle 106 and perpendicular to the pattern of lamps 102 and surface of the fixture 110. The embodiment shown is intended to be mounted to a ceiling or affixed to another device to allow for the lamps to create a Barrier Zone 150 towards a ceiling or upper air space of a room. It should be appreciated that other embodiments can be mounted to poles or hang from poles or other objects affixed to a wall and achieve the same desired effect of creating a Barrier Zone 150. The embodiment shown further includes support tabs 109 affixed to a portion or surface of the fixture 101 and further include support wire 111 or other material extending from the support tab 109 to an upper support body 112 having complimentary support tabs 114 or portions or surfaces to accept a terminal end of the support wire 113 or other material. The support 115 extending through and from the lamp receptacle 106 can extend through and to the upper support body 112 to provide an attachment or mounting point for the system to a ceiling or other object. It should be appreciated that the support 115 can comprise or provide a route for electrical wire for power delivery to the system 100. It should also be appreciated that the electrical wire or power delivery could be achieved via one or more of the support wires 111 or other material.

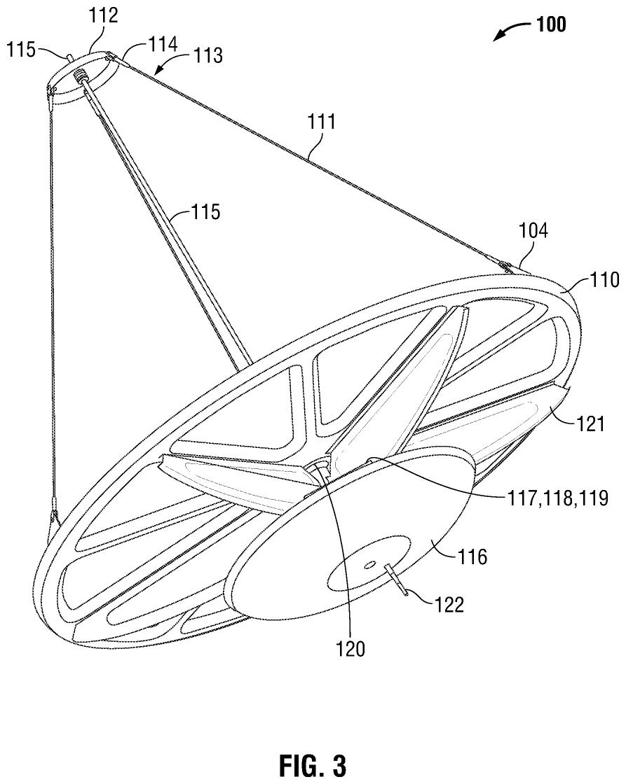

FIG. 3 illustrates one embodiment of the present invention. The system 100 depicted in this embodiment includes a fixture 101 having a circular shape for housing the lamps 102 (FIG. 2). It should be appreciated that the fixture can be formed in various shapes whether for functional or aesthetic purposes. The embodiment shown includes a support 115, e.g. wire or column, extending through and from the lamp receptacle 106 and perpendicular to the pattern of lamps 102 and surface of the fixture 110. The embodiment shown is intended to be mounted to a ceiling or affixed to another device to allow for the lamps to create a Barrier Zone 150 (FIG. 1) towards a ceiling or upper air space of a room. It should be appreciated that other embodiments can be mounted to poles or hang from poles or other objects affixed to a wall and achieve the same desired effect of creating a Barrier Zone 150 (FIG. 1). The embodiment shown further includes support tabs 109 affixed to a portion or surface of the fixture 101 and further include support wire 111 or other material extending from the support tab 109 to an upper support body 112 having complimentary support tabs 114 or portions or surfaces to accept a terminal end of the support wire 113 or other material. The support 115 extending through and from the lamp receptacle 106 can extend through and to the upper support body 112 to provide an attachment or mounting point for the system to a ceiling or other object. It should be appreciated that the support 115 can comprise or provide a route for electrical wire for power delivery to the system 100. It should also be appreciated that the electrical wire or power delivery could be achieved via one or more of the support wires 111 or other material. The system 100 as shown in this embodiment includes a lower body 116 disposed below the fixture wherein the sensors 117, processors 118, lamp drivers 119, antenna 122 and other electrical components can be housed separate from the lamps 102. It should be appreciated that antenna 122 could be internal or protruding from an exterior surface of the system. It should also be appreciated that antenna 122 could be a wi-fi antenna, cellular antenna, a sensor (as described herein), or combination of components for taking measurements and/or communications, such as Wi-Fi, cloud networking, or Internet access to a remote server or computer. In this embodiment, the lower body 116 is affixed to the fixture through an intermediate support 120 having a plurality of support fins 121 extending away from the intermediate support 120 in a “radial” pattern. The support fins 121 can provide rigidity to the fixture, act as heat sinks, and/or provide aesthetic appeal.

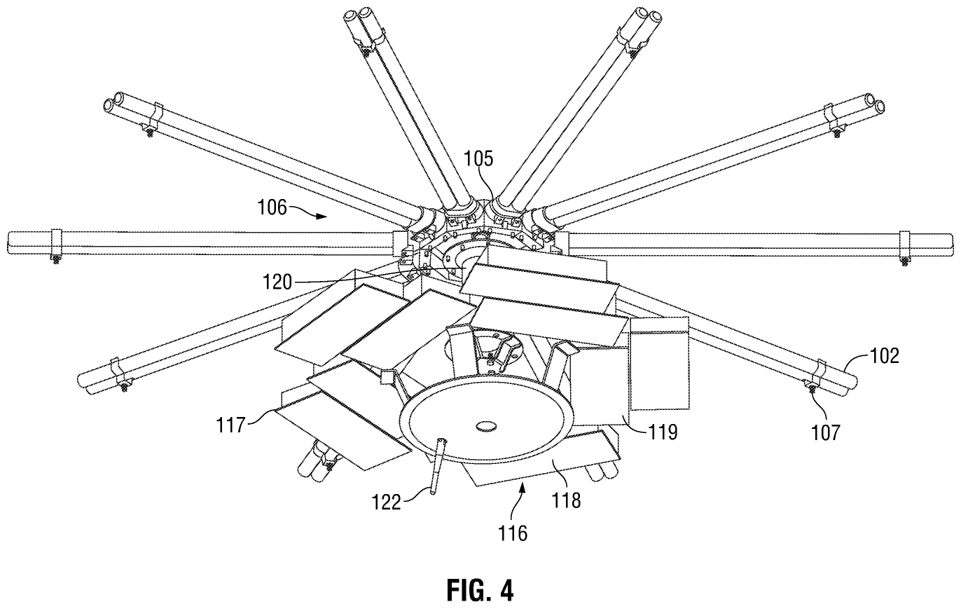

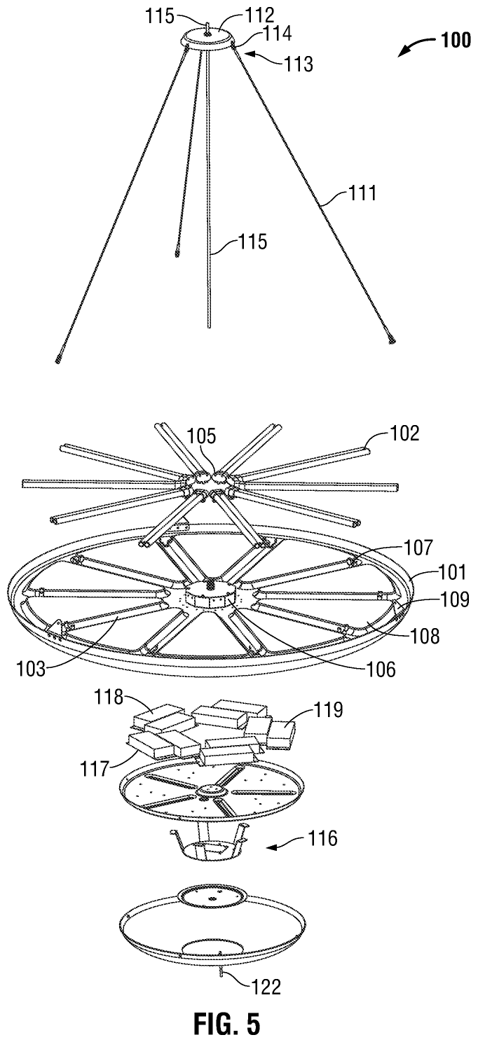

FIG. 4 shows a detail view of one embodiment of the invention. Shown are lamps 102 having a terminal end 104 (FIG. 2) with a socket 105 to be plugged into a lamp receptacle 106. The other end of the lamp may be secured with a clip 107, as shown, or rest on top of a portion of the fixture. The lamp receptacle 106 is disposed at or near the center of the fixture in this embodiment to create a “sunburst” or “radial” pattern of lamps 102. It should be appreciated that various configurations of lamps 102, lamp receptacles 106, and fixtures 101 (FIGS. 1 and 2) can be made to achieve varying coverage of the Barrier Zone 150 (FIG. 1) and/or for aesthetic purposes. The embodiment as shown in this drawing includes a lower body 116 disposed below where the fixture would typically be disposed, wherein the sensors 117, processors 118, lamp drivers 119, antenna 122 and other electrical components can be housed separate from the lamps 102. It should be appreciated that antenna 122 could be internal or protruding from an exterior surface of the system. It should also be appreciated that antenna 122 could be a wi-fi antenna, cellular antenna, a sensor (as described herein), or combination of components for taking measurements and/or communications, such as Wi-Fi, cloud networking, or Internet access to a remote server or computer. FIG. 5 illustrates one embodiment of the present invention using an exploded component view. The system 100 depicted in this embodiment includes a fixture 101 having a circular shape for housing the lamps 102. It should be appreciated that the fixture can be formed in various shapes whether for functional or aesthetic purposes. The fixture 101 can include indentions or tracks 103 wherein one or more lamps 102 can be recessed. The fixture 101 depicted includes lamps 102 having a terminal end 104 with a socket 105 to be plugged into a lamp receptacle 106. The other end of the lamp may be secured with a clip 107, as shown, or rest on top of a portion of the fixture. The lamp receptacle 106 is disposed at or near the center of the fixture in this embodiment to create a “sunburst” or “radial” pattern of lamps 102. It should be appreciated that various configurations of lamps 102, lamp receptacles 106, and fixtures 101 can be made to achieve varying coverage of the Barrier Zone 150 (FIG. 1) and/or for aesthetic purposes. Preferably, the fixture 101 includes one or more reflectors 108 under the one or more lamps 102 to reflect additional UV light away from the surface of the fixture 101. The use of reflectors 108 helps avoid any wastage of the UV light by redirecting additional light toward the Barrier Zone 150 (FIG. 1), that might otherwise be absorbed by the surface of the fixture 110. The embodiment shown includes a support 115, e.g. wire or column, extending through and from the lamp receptacle 106 and perpendicular to the pattern of lamps 102 and surface of the fixture 110. The embodiment shown is intended to be mounted to a ceiling or affixed to another device to allow for the lamps to create a Barrier Zone 150 (FIG. 1) towards a ceiling or upper air space of a room. It should be appreciated that other embodiments can be mounted to poles or hang from poles or other objects affixed to a wall and achieve the same desired effect of creating a Barrier Zone 150 (FIG. 1). The embodiment shown further includes support tabs 109 affixed to a portion or surface of the fixture 101 and further include support wire 111 or other material extending from the support tab 109 to an upper support body 112 having complimentary support tabs 114 or portions or surfaces to accept a terminal end of the support wire 113 or other material. The support 115 extending through and from the lamp receptacle 106 can extend through and to the upper support body 112 to provide an attachment or mounting point for the system to a ceiling or other object. It should be appreciated that the support 115 can comprise or provide a route for electrical wire for power delivery to the system 100. It should also be appreciated that the electrical wire or power delivery could be achieved via one or more of the support wires 111 or other material. The system 100 as shown in this embodiment includes a lower body 116 disposed below the fixture wherein the sensors 117, processors 118, lamp drivers 119, antenna 122 and other electrical components can be housed separate from the lamps 102. It should be appreciated that antenna 122 could be internal or protruding from an exterior surface of the system. It should also be appreciated that antenna 122 could be a wi-fi antenna, cellular antenna, a sensor (as described herein), or combination of components for taking measurements and/or communications, such as Wi-Fi, cloud networking, or Internet access to a remote server or computer. In this embodiment, the lower body 116 is affixed to the fixture through an intermediate support 120 having a plurality of support fins 121 extending away from the intermediate support 120 in a “radial” pattern. The support fins 121 can provide rigidity to the fixture, act as heat sinks, and/or provide aesthetic appeal.

While preferred embodiments have been shown and described, modifications thereof can be made by one skilled in the art without departing from the scope or teaching herein. The embodiments described herein are exemplary only and are not limiting. Many variations and modifications of the system and apparatus are possible and will become apparent to those skilled in the art once the above disclosure is fully appreciated. For example, the relative dimensions of various parts, the materials from which the various parts are made, and other parameters can be varied. Accordingly, it is intended that the following claims be interpreted to embrace all such variations and modifications.

What is claimed is the following:

1. An improved pathogen deactivating ultraviolet light system comprising:
an upper mounting body having a substantially circular shape and having an upper surface;
a plurality of mounting brackets disposed on said upper surface of said upper mounting body;
a plurality of ultraviolet lamps fixedly attached to said plurality of mounting brackets;
a plurality of reflectors disposed on the upper surface of said upper mounting body and under said plurality of ultraviolet lamps;
wherein, said plurality of ultraviolet lamps are arranged in a radial pattern wherein a proximal end of each ultraviolet lamp begins near a middle point of said upper mounting body and a distal end of each ultraviolet lamp is disposed toward an exterior edge formed by a perimeter of said upper mounting body;
wherein, said plurality of ultraviolet lamps when energized produce a Barrier Zone upward and away from said upper surface of the upper mounting body, wherein said Barrier Zone is the active zone for the plurality of ultraviolet lamps to deactivate one or more pathogens; and
wherein, said upper mounting body has a collar that effectively blocks any UV light emitted by the ultraviolet lamps from reaching any humans below the plane of the upper body of the assembly and that may be modified to block UP light projection on to specific areas above the plane of the upper body of the assembly.

2. An improved pathogen deactivation ultraviolet light system comprising:
a fixture having a surface, wherein said surface comprises one or more reflective surfaces, wherein said fixture further comprises a collar;
a lamp receptacle for receiving a plurality of ultraviolet lamps, said lamp receptacle having sockets for receiving a terminal end of each of said plurality of ultraviolet lamps, wherein said plurality of ultraviolet lamps are disposed below said collar;
a lower body comprising a plurality of support fins, sensors, processors, and lamp drivers disposed within a cavity formed by the lower body; and
a power source operatively connected to the system to provide power to said lamp receptacle, said plurality of ultraviolet lamps, said sensors, said processors, and said lamp drivers.

3. A method for deactivating pathogens comprising the steps of:
creating a barrier zone by powering a plurality of ultraviolet lamps using a system comprising:
a fixture having a surface, wherein said surface comprises one or more reflective surfaces;
a lamp receptacle for receiving the plurality of ultraviolet lamps, said lamp receptacle having sockets for receiving a terminal end of each of said plurality of ultraviolet lamps;
a lower body comprising a plurality of support fins, sensors, processors, and lamp drivers disposed within a cavity formed by the lower body; and
a power source operatively connected to the system to provide power to said lamp receptacle, said plurality of ultraviolet lamps, said sensors, said processors, and said lamp drivers.
